(19) United States
(12) Patent Application Publication
(10) Pub. No.: US 2025/0346620 A1
(43) Pub. Date: Nov. 13, 2025

(54) METHODS FOR SYNTHESIS OF PERACETYLGALACTOSAMINE-1-PENTANOIC ACID

(71) Applicant: DICERNA PHARMACEUTICALS, INC., Lexington, MA (US)

(72) Inventors: Kurt Edric VAGLE, Longmont, CO (US); Sayantan BHADURI, Longmont, CO (US)

(21) Appl. No.: 18/860,285

(22) PCT Filed: Apr. 27, 2023
(86) PCT No.: PCT/US2023/020104

§ 371 Date: Oct. 25, 2024

Related U.S. Application Data
(60) Provisional application No. 63/335,830, filed on Apr. 28, 2022.

(51) Int. Cl. C07H 15/04; C07H 1/00

(52) U.S. Cl. CPC C07H 15/04; C07H 1/00

(57) ABSTRACT

The disclosure provides methods for synthesis of peracetylgalactosamine-1-pentanoic acid, also called peracetylated D-galactosamine C5 linker or GalNAc C5 linker, using a vegetal source as a starting material, such as vegetal-sourced D-glucosamine or D-glucosamine hydrochloride. Also provided are methods for purifying the peracetylgalactosamine-1-pentanoic acid, or GalNAc C5 linker, thus produced so that the end product comprises fewer impurities.

### CROSS REFERENCE TO RELATED APPLICATIONS

This application claims priority to U.S. Provisional Application No. 63/335,830, filed 28 Apr. 2022, the entire contents of which are hereby incorporated by reference.

### FIELD

The present disclosure relates generally to methods for synthesis of peracetylgalactosamine-1-pentanoic acid, also called peracetylated D-galactosamine C5 linker or GalNAc C5 linker, using a vegetal source as a starting material, such as vegetal-sourced D-glucosamine or D-glucosamine hydrochloride.

### BACKGROUND

In recent years, approaches have been developed to use nucleic acid molecules in therapy. A main challenge in realizing the full potential of nucleic acid therapeutics is efficient delivery of nucleic acid molecules into targeted tissues and cells in an efficient and therapeutically efficacious way. N-acetylgalactosamine (GalNAc) is a well-defined liver-targeted moiety benefiting from its high affinity with the asialoglycoprotein receptor (ASGPR). By conjugating GalNAc to oligonucleotides or incorporating GalNAc into a certain delivery system as a targeting moiety, GalNAc has achieved compelling successes in the development and delivery of nucleic acid therapeutics in recent years.

In the manufacturing process of GalNAc conjugates, such as 2′-O-GalNAc-modified adenosine (AdemA-GalNAc) and 2′-O-GalNAc-modified guanosine (AdemG-GalNAc), peracetylgalactosamine-1-pentanoic acid, also called peracetylated D-galactosamine C5 linker or GalNAc C5 linker, is an important raw material. FIG. 1 represents a schematic showing a traditional method for synthesizing this GalNAc C5 linker using D-galactosamine hydrochloride as starting material, which has been available only from animal sources, such as chicken bones or other avian animals. However, it is desirable to obtain the GalNAc C5 linker free of components derived from any animal sources.

In addition, there has been a persistent impurity species, namely peracetylgalactosamine-1-butanoic acid or “M-14” (“GalNAc C4 Linker” in FIG. 1) in the amidite products, detectable up to 1% on HPLC (High Performance Liquid Chromatography), which may be incorporated into drug substances. This impurity is believed to be derived from an impurity of 4-hexen-1-ol present in the raw material 5-hexen-1-ol used in the synthesis of GalNAc C5 linker. It is desirable to reduce the level of the M-14 impurity in the final GalNAc C5 linker product, preferably to a level below detection limit, which is 0.05% by ICH guidelines.

Accordingly, there is still a need in the art to develop a new synthetic method for synthesis of GalNAc C5 linker from a non-animal sourced starting material with simple operation, easy purification of products, and high chemical yield.

### SUMMARY

The present disclosure is directed to methods for synthesis of peracetylgalactosamine-1-pentanoic acid, also called peracetylated D-galactosamine C5 linker or GalNAc C5 linker, using a vegetal source as a starting material, such as vegetal-sourced D-glucosamine or D-glucosamine hydrochloride. GalNAc C5 linker is traditionally synthesized from galactosamine or derivatives thereof, which can only be economically isolated from animal sources. Glucosamine is readily available from non-animal sources and is much more economical and more sustainable than galactosamine. The methods disclosed herein allows for the cost-effective synthesis of GalNAc conjugated straight chain acids that are nearly 100% pure and free of persistent and difficult to remove impurities, like peracetylgalactosamine-1-butanoic acid, that may be carried through to subsequent synthetic products as a critical impurity where GaNAc C5 linker is incorporated via esterification or amidation reactions.

In a first aspect, this disclosure provides a method for synthesizing peracetylgalactosamine-1-pentanoic acid, or peracetylated D-galactosamine C5 Linker (GalNAc C5 Linker), comprising using a vegetal source, such as vegetal D-glucosamine or vegetal D-glucosamine hydrochloride, as a starting material and using a synthetic route that introduces glycosylation of a lipophilic, masked carboxylate, followed by C4 inversion.

In a second aspect, this disclosure provides a method for producing peracetylgalactosamine-1-pentanoic acid (Formula A):

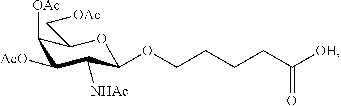

the method comprising subjecting D-glucosamine hydrochloride to glycosylation followed by C4 inversion, leading to the production of peracetylgalactosamine-1-pentanoic acid (Formula A). In some embodiments, the method disclosed herein comprises the synthetic route illustrated in FIG. 3.

In a third aspect, this disclosure provides a method for producing peracetylgalactosamine-1-pentanoic acid (Formula A):

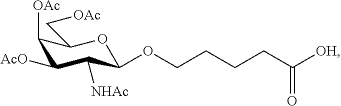

wherein the method comprises using a vegetal source, such as vegetal D-glucosamine or vegetal D-glucosamine hydrochloride, as a starting material and the peracetylgalactosamine-1-pentanoic acid (Formula A) contains less than 0.05% of peracetylgalactosamine-1-butanoic acid, as analyzed by high-performance liquid chromatography (HPLC). In some embodiments, the 5-hexen-1-ol used for the glycosylation of a derivatized D-glucosamine intermediate contains less than about 0.2% of 4-hexen-1-ol.

In all aspects, the disclosed methods further comprise a step of re-crystallization of the final reaction product, i.e., peracetylgalactosamine-1-pentanoic acid.

### BRIEF DESCRIPTION OF THE DRAWINGS

The accompanying drawings, which are incorporated in and constitute a part of this specification, illustrate certain embodiments, and together with the written description, serve to explain certain principles of the methods and devices disclosed herein.

FIG. 1 depicts a synthetic scheme of GalNAc C5 linker from animal sourced galactosamine.

FIG. 2A-2B depict two design strategies for synthesizing GalNAc C5 linker using vegetal glucosamine as a starting material. FIG. 2A: Design strategy “Route 1” introduces glycosylation early in the route to allow easier synthetic workup and to minimize changes to existing manufacturing process. FIG. 2B: Design strategy “Route 2” allows making galactosamine as a stock-up intermediate and makes it possible to use the resultant galactosamine as a starting material for GalNAc C5 linker or other galactosamine products.

FIG. 3 depicts a synthetic scheme of GalNAc C5 linker using vegetal D-glucosamine hydrochloride as a starting material via design strategy “Route 1.”

FIG. 4 depicts the HPLC analysis of glycosylated Intermediate 4 and Intermediate 7 synthesized by design strategy “Route 1.”

FIG. 5 depicts the HPLC comparison of Intermediate 8 synthesized by design strategy “Route 1” with synthetic reference material synthesized using animal sourced D-galactosamine.

FIG. 6 depicts the ¹H-NMR comparison of GalNAc C5 linker synthesized by design strategy “Route 1” with GalNAc C5 linker synthesized using animal sourced D-galactosamine (Top: GalNAc C5 linker from vegetal D-glucosamine; Bottom: GalNAc C5 linker from animal sourced D-galactosamine).

FIG. 7 depicts the HPLC analysis of impurity M-14 in GalNAc C5 linkers synthesized by design strategy “Route 1” using 5-hexen-1-ol with different amounts of 4-hexen-1-ol (low content vs. higher content).

FIG. 8 depicts a synthetic scheme of GalNAc C5 linker using vegetal glucosamine as a starting material via design strategy “Route 2.”

FIG. 9 depicts an alternative synthetic scheme of GalNAc C5 linker using vegetal glucosamine as a starting material via design strategy “Route 2.”

### DETAILED DESCRIPTION

In the published methods of an N-acetyl glucosamine (GlcNAc) to N-Acetyl galactosamine (GalNAc) conversion, the synthetic route achieves C4 inversion followed by further modification of the sugar moiety to give GalNAc C5 linker. Synthesis of the carbohydrate intermediates are known, but isolation of the carbohydrate components of known routes introduces difficulty in purification of undesired isomers and isolation of highly water-soluble products, making the process challenging to scale in an economical fashion. The synthetic sugar is then glycosylated with an appropriate masked carboxylate after isolation of the desired carbohydrate. Along with difficulty in isolation of the carbohydrate component, glycosylation with common sources of masked carboxylates has the potential to introduce known impurities that are not readily removed prior to immediately converting to the desired GalNAc C5 linker. Similarly, the use of animal derived galactosamine accomplishes glycosylation immediately prior to conversion of the masked carboxylate to the desired GalNAc C5 linker. The methods disclosed herein employ a synthetic route that introduces glycosylation with a lipophilic alkyl chain early in the process, for example, by introducing a 5-hexen-1-ol or a derivative thereof (e.g., 5-hexen-1-ol optionally substituted at the 6 position) at anomeric carbon prior to C4 inversion. Contrary to the known methods, the disclosed methods are much more efficient, in part, because the glycosidic “protecting group” serves as a purification handle throughout the synthesis, as well as a masked carboxylate that is directly converted to the final linker product.

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods and/or to make and use the disclosed devices. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the term “in some embodiments” refers to embodiments of all aspects of the disclosure, unless the context clearly indicates otherwise.

The terms “peracetylgalactosamine-1-pentanoic acid,” “peracetylated D-galactosamine C5 linker” and “GalNAc C5 linker” are used interchangeably herein and refer to a compound of Formula A:

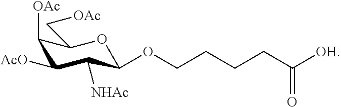

### Design Strategy “Route 1”

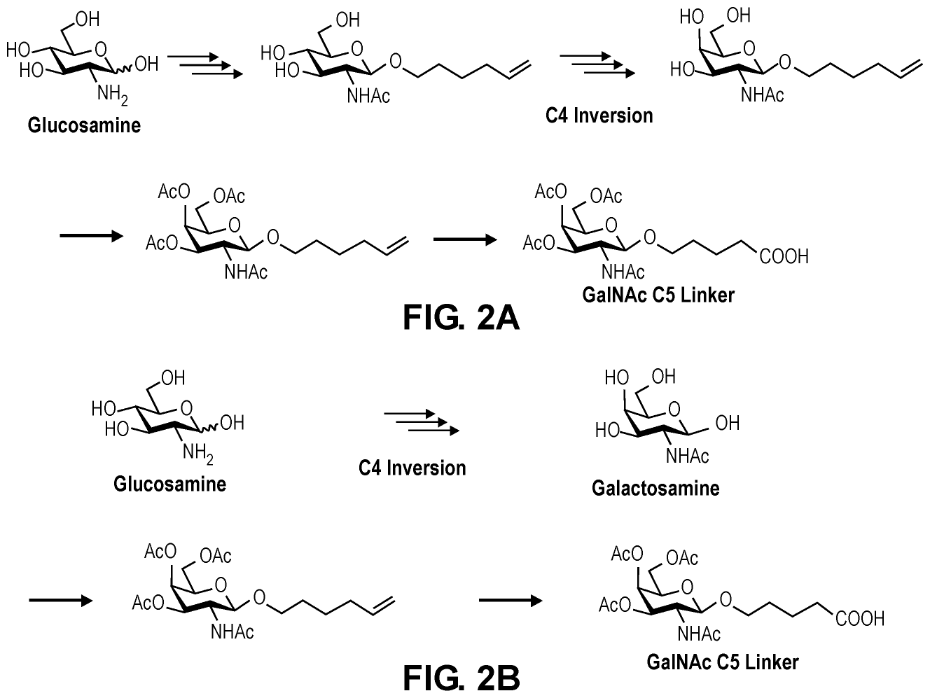

In one aspect, peracetylgalactosamine-1-pentanoic acid, or peracetylated D-galactosamine C5 Linker (GalNAc C5 Linker), of Formula A can be synthesized from a vegetal source, such as vegetal D-glucosamine hydrochloride, as a starting material via so-called design strategy “Route 1,” which introduces glycosylation early in the process followed by C4 inversion, as depicted in FIG. 2A. This design strategy “Route 1” uses D-glucosamine hydrochloride as the starting material for producing peracetylgalactosamine-1-pentanoic acid of Formula A and comprises at least the following steps:
a) subjecting D-glucosamine hydrochloride to acetylation followed by glycosylation with 5-hexen-1-ol to obtain a derivatized D-glucosamine intermediate of formula 3:

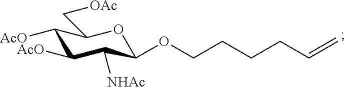

b) subjecting the derivatized D-glucosamine intermediate of formula 3 to hydrolysis followed by selective acetylation to obtain a derivatized D-glucosamine intermediate of formula 5:

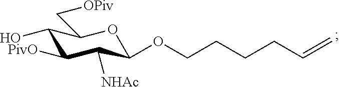

c) subjecting the derivatized D-glucosamine intermediate of formula 5 to triflation leading to an inversion of the chiral configuration at C4 position to obtain a derivatized D-galactosamine intermediate of formula 6:

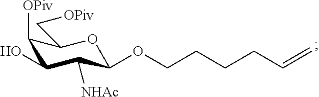

and
d) subjecting the derivatized D-galactosamine intermediate of formula 6 to hydrolysis followed by acetylation and oxidation thereby producing the peracetylgalactosamine-1-pentanoic acid of Formula A:

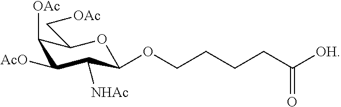

In some embodiments, the acetylation of D-glucosamine hydrochloride is performed in the presence of acetic anhydride. In some embodiments, the acetylation of D-glucosamine hydrochloride by acetic anhydride is catalyzed by pyridine. In other embodiments, the acetylation of D-glucosamine hydrochloride by acetic anhydride is catalyzed by pyridine and 4-dimethylaminopyridine (DMAP). The acetylation of D-glucosamine hydrochloride by acetic anhydride leads to the production of a derivatized D-glucosamine intermediate of formula 1:

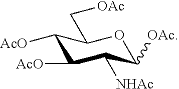

In some embodiments, the derivatized D-glucosamine intermediate of formula 1 is extracted using ethyl acetate (EA) and the extract product is reacted with trimethylsilyl trifluoromethanesulfonate (TMSOTf). In some embodiments, this reaction with TMSOTf is performed in the presence of acetonitrile (CAN). The reaction of the derivatized D-glucosamine intermediate of formula 1 with TMSOTf leads to the production of a derivatized D-glucosamine intermediate of formula 2:

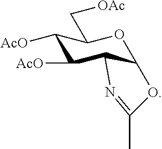

In some embodiments, the derivatized D-glucosamine intermediate of formula 2 is extracted using dichloromethane (DCM) and subjected to glycosylation by 5-hexen-1-ol. In some embodiments, the glycosylation of the derivatized D-glucosamine intermediate of formula 2 is performed in the presence of TMSOTf. The glycosylation of the derivatized D-glucosamine intermediate of formula 2 by 5-hexen-1-ol leads to the production of the derivatized D-glucosamine intermediate of formula 3:

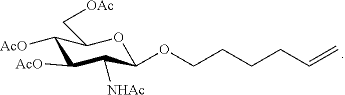

In some embodiments, a derivative of 5-hexen-1-ol cane be used instead of 5-hexen-1-ol in the glycosylation of the derivatized D-glucosamine intermediate of formula 2. A derivative of 5-hexen-1-ol can be a 5-hexen-1-ol optionally substituted at the 6 position, such as one with the following structure:

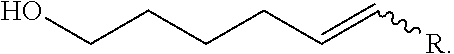

wherein R is a protected or unprotected —OH, —Oalkyl or —NH2, a —COOH, an ester, an aryl, a heteroaryl or a heterocycle.

In some embodiments, the derivatized D-glucosamine intermediate of formula 3 is hydrolyzed by sodium methoxide. In some embodiments, the hydrolysis of the derivatized D-glucosamine intermediate of formula 3 by sodium methoxide is performed in methanol. In some embodiments, the hydrolysis of the derivatized D-glucosamine intermediate of formula 3 liberates three hydroxy groups at C3, C4 and C6 positions. The hydrolysis of the derivatized D-glucosamine intermediate of formula 3 leads to the production of a derivatized D-glucosamine intermediate of formula 4:

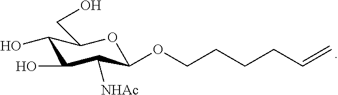

To obtain the derivatized D-glucosamine intermediate of formula 5, the derivatized D-glucosamine intermediate of formula 4 is subjected to selective protection as pivaloyl esters at C3 and C6 positions. In some embodiments, this selective protection is conducted in the presence of pivaloyl chloride. In some embodiments, this selective protection by pivaloyl chloride is performed in pyridine.

In some embodiments, the derivatized D-glucosamine intermediate of formula 5 is extracted using DCM and water, and the extracted product is subjected to triflation followed by a SN2 migration of a pivaroyl group from C3 to C4 position (i.e., C4 inversion) to obtain the derivatized D-galactosamine intermediate of formula 6:

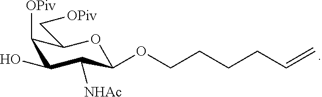

In some embodiments, this triflation followed by C4 inversion is mediated by triflic anhydride. In such embodiments, the oxygen atom at the C4 position, also referred to as the “O-4 position,” is activated by triflate to form an intermediate O-4 triflate followed by an intramolecular attack of the intermediate O-4 triflate by the pivaloyl group at the C3 position, also referred to as the “O-3-pivaloyl group,” resulting in an in-situ displacement of the triflate to yield the derivatized D-galactosamine intermediate of formula 6. In some embodiments, this triflation followed by C4 inversion is performed in the presence of pyridine and DCM.

In some embodiments, the derivatized D-galactosamine intermediate of formula 6 is extracted using ethyl acetate (EA) and the extracted product is subjected to hydrolysis by sodium methoxide. In some embodiments, the hydrolysis of the derivatized D-galactosamine intermediate of formula 6 is performed in methanol. The hydrolysis of the derivatized D-galactosamine intermediate of formula 6 leads to the production of a derivatized D-galactosamine intermediate of formula 7:

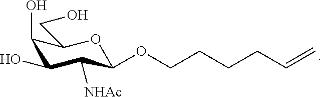

In some embodiments, the derivatized D-galactosamine intermediate of formula 7 is concentrated and dried, and the dried product is subjected to acetylation by acetic anhydride. In some embodiments, the acetylation of the derivatized D-galactosamine intermediate of formula 7 by acetic anhydride is catalyzed by pyridine. In other embodiments, the acetylation of the derivatized D-galactosamine intermediate of formula 7 by acetic anhydride is catalyzed by pyridine and DMAP. The acetylation of the derivatized D-galactosamine intermediate of formula 7 leads to the production of a derivatized D-galactosamine intermediate of formula 8:

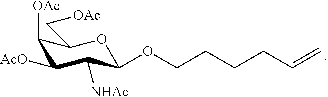

In some embodiments, the derivatized D-galactosamine intermediate of formula 8 is extracted using DCM and water and the extracted product is subjected to oxidation by sodium periodate. In some embodiments, the oxidation of the derivatized D-galactosamine intermediate of formula 8 is catalyzed by ruthenium chloride. In some embodiments, the oxidation of the derivatized D-galactosamine intermediate of formula 8 is performed in the presence of acetonitrile, DCM, and water. The oxidation of the derivatized D-galactosamine intermediate of formula 8 leads to the production of the peracetylgalactosamine-1-pentanoic acid of Formula A. In some embodiments, the peracetylgalactosamine-1-pentanoic acid of Formula A is extracted using DCM.

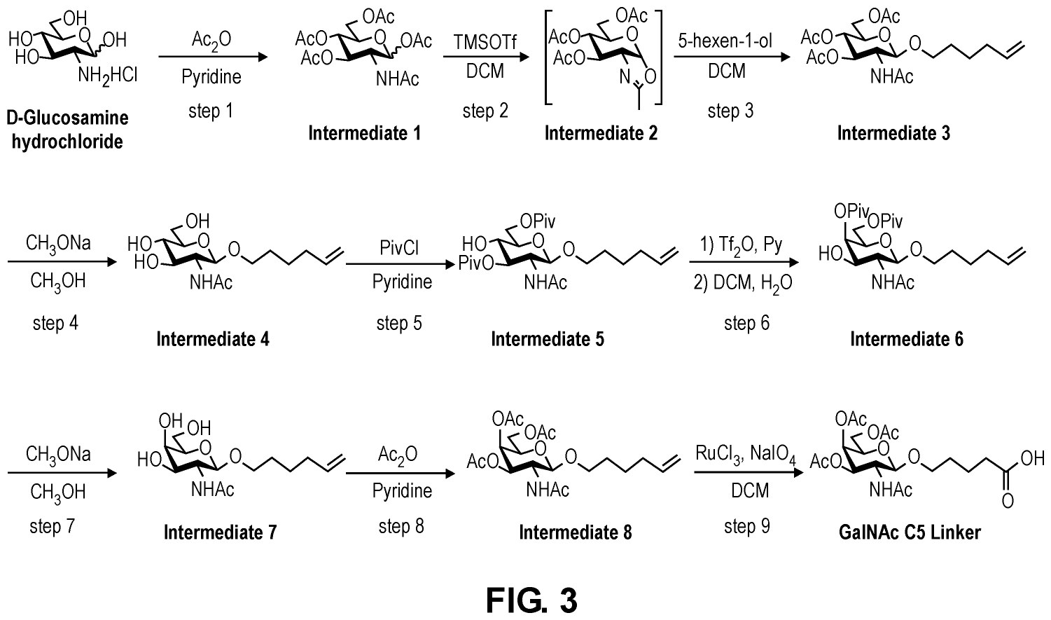

In some embodiments, the synthetic route of the disclosed methods based on design strategy “Route 1” is reflected in FIG. 3 and comprises:
a) subjecting D-glucosamine hydrochloride to acetylation by acetic anhydride to obtain the derivatized D-glucosamine intermediate of formula 1;
b) reacting the derivatized D-glucosamine intermediate of formula 1 with TMSOTf to obtain the derivatized D-glucosamine intermediate of formula 2;
c) subjecting the derivatized D-glucosamine intermediate of formula 2 to glycosylation by 5-hexen-1-ol to obtain the derivatized D-glucosamine intermediate of formula 3;
d) subjecting the derivatized D-glucosamine intermediate of formula 3 to hydrolysis by sodium methoxide to obtain the derivatized D-glucosamine intermediate of formula 4;
e) reacting the derivatized D-glucosamine intermediate of formula 4 with pivaloyl chloride to obtain a derivatized D-glucosamine intermediate of formula 5;
f) reacting the derivatized D-glucosamine intermediate of formula 5 with triflic anhydride to obtain a derivatized D-galactosamine intermediate of formula 6, wherein the derivatized D-glucosamine intermediate of formula 5 is activated at O-4 position by triflate to form an intermediate O-4 triflate followed by an intramolecular attack of the intermediate O-4 triflate by the O-3-pivaloyl group resulting in an in-situ displacement of the triflate to yield the derivatized D-galactosamine intermediate of formula 6;
g) subjecting the derivatized D-galactosamine intermediate of formula 6 to hydrolysis by sodium methoxide to obtain a derivatized D-galactosamine intermediate of formula 7;
h) subjecting the derivatized D-galactosamine intermediate of formula 7 to acetylation by acetic anhydride to obtain the derivatized D-galactosamine intermediate of formula 8; and
i) subjecting the derivatized D-galactosamine intermediate of formula 8 to oxidation by sodium periodate thereby producing the peracetylgalactosamine-1-pentanoic acid of Formula A.

In some embodiments, after completing a reaction step but prior to subjecting to the subsequent reaction step, the derivatized D-glucosamine intermediate or derivatized D-galactosamine intermediate may be precipitated using a crystallization solvent and then filtered. In some embodiments, the crystallization solvent is a low-polarity solvent such as n-heptane and/or cyclohexane. In some embodiments, the crystallization solvent is n-heptane. In some embodiments, the crystallization solvent is cyclohexane.

### Starting Materials

The starting material for the synthetic route based on design strategy “Route 1” is D-glucosamine, which can be derived from any source, but preferably vegetal sources. The D-glucosamine may be in the form of a salt, such as hydrochloride (i.e., D-glucosamine hydrochloride). In some embodiments, therefore, the D-glucosamine hydrochloride used in the disclosed synthetic methods is derived from a plant source. Vegetal sources of D-glucosamine hydrochloride may include, but are not limited to, fungi, such as Aspergillus niger, and fermenting corn (“Another vegetarian glucosamine launched in US,” Nutrangredients-USA.com, Jan. 25, 2008). In some embodiments, the D-glucosamine hydrochloride used in the disclosed synthetic methods is derived from Aspergillus niger. In some embodiments, the D-glucosamine hydrochloride used in the disclosed synthetic methods is derived from fermenting corn.

The starting material for the synthetic route based on design strategy “Route 2” is N-acetylglucosamine, which can be derived from any source, but preferably vegetal sources. In some embodiments, therefore, the N-acetylglucosamine used in the disclosed synthetic methods is derived from a plant source. N-acetylglucosamine is known to be the major component of the cell walls of most fungi. In some embodiments, the N-acetylglucosamine used in the disclosed synthetic methods is derived from fungi.

### Elimination of Impurities

In the traditional synthetic route that uses galactosamine as a starting material to make GalNAc C5 linker, there has been a persistent impurity species, namely peracetylgalactosamine-1-butanoic acid (GalNAc C4 linker or “M-14”). This impurity is known to be derived from an impurity of 4-hexen-1-ol present in the raw material 5-hexen-1-ol used for glycosylation. The level of “M-14” impurity in the final GalNAc C5 linker product can be reduced by using 5-hexen-1-ol raw material containing less impurity 4-hexen-1-ol. In some embodiments therefore, the 5-hexen-1-ol used for glycosylation in the disclosed methods contains less than about 0.3% impurity 4-hexen-1-ol. In some embodiments, the 5-hexen-1-ol used for glycosylation in the disclosed methods contains less than about 0.2% impurity 4-hexen-1-ol. In some embodiments, the 5-hexen-1-ol used for glycosylation in the disclosed methods contains less than about 0.15% impurity 4-hexen-1-ol. In some embodiments, the 5-hexen-1-ol used for glycosylation in the disclosed methods contains less than about 0.1% impurity 4-hexen-1-ol. In some embodiments, the 5-hexen-1-ol used for glycosylation in the disclosed methods contains less than about 0.05% impurity 4-hexen-1-ol. In some embodiments, the 5-hexen-1-ol used for glycosylation in the disclosed methods contains less than about 0.03% impurity 4-hexen-1-ol. In some embodiments, the 5-hexen-1-ol used for glycosylation in the disclosed methods contains less than about 0.01% impurity 4-hexen-1-ol.

It has been found that the level of “M-14” impurity, as well as other impurities, in the final GalNAc C5 linker product can be further improved by using an isolation and purification process which includes a first partition leading to an alkaline aqueous phase and a second partition leading to an acidic aqueous phase. Thus, in some embodiments, the peracetylgalactosamine-1-pentanoic acid of Formula A synthesized by any of the disclosed methods is purified by:
a) subjecting a reaction mixture comprising the peracetylgalactosamine-1-pentanoic acid of Formula A to a first partition leading to an alkaline aqueous phase and a first organic phase;
b) subjecting the alkaline aqueous phase to a second partition leading to an acidic aqueous phase and a second organic phase; and
c) combining the first organic phase and the second organic phase followed by precipitating the peracetylgalactosamine-1-pentanoic acid of Formula A.

In some embodiments, the first partition leading to an alkaline aqueous phase is performed by adjusting the pH of the reaction mixture comprising the peracetylgalactosamine-1-pentanoic acid of Formula A to about pH 8. Any alkali may be used to adjust the pH of the reaction mixture comprising the peracetylgalactosamine-1-pentanoic acid of Formula A. In some embodiments, the pH of the reaction mixture comprising the peracetylgalactosamine-1-pentanoic acid of Formula A is adjusted to about pH 8 using sodium bicarbonate.

In some embodiments, the second partition leading to an acidic aqueous phase is performed by adjusting the pH of the alkaline aqueous phase to about pH 2. Any acid commonly used in chemical synthesis may be used to adjust the pH of the alkaline aqueous phase. Exemplary acids may include, but not limited to, acetic acid, boric acid, citric acid, hydrochloric acid, and sulfuric acid. In some embodiments, the pH of the alkaline aqueous phase is adjusted to about pH 2 using citric acid.

In some embodiments, the precipitation of the peracetylgalactosamine-1-pentanoic acid of Formula A from the combined first organic phase and the second organic phase is performed in the presence of methyl tertiary-butyl ether (MTBE).

Additional steps, such as filtration and washing, may be included before or after each partition. In some embodiments therefore, the peracetylgalactosamine-1-pentanoic acid of Formula A synthesized by any of the disclosed methods is purified by:
a) filtering a reaction mixture comprising the peracetylgalactosamine-1-pentanoic acid of Formula A to obtain a filtered solution;
b) adjusting pH of the filtered solution to about pH 8;
c) separating phases to obtain an alkaline aqueous phase and a first organic phase;
d) adjusting pH of the alkaline aqueous phase to about pH 2;
e) separating phases to obtain an acidic phase and a second organic phase; and
e) combining the first organic phase and the second organic phase and adding methyl tert-butyl ether (MTBE) to allow precipitates to form, wherein the precipitates comprise the peracetylgalactosamine-1-pentanoic acid of Formula A.

In some embodiments, the pH of the filtered solution is adjusted to about pH 8 using sodium bicarbonate. In some embodiments, the pH of the alkaline aqueous phase is adjusted to about pH 2 using citric acid.

The level of impurities contained in the peracetylgalactosamine-1-pentanoic acid of Formula A obtained by any of the disclosed methods may be determined using any methods known in the art, such as HPLC. In some embodiments, the peracetylgalactosamine-1-pentanoic acid of Formula A obtained from the disclosed methods comprises less than 0.05% of peracetylgalactosamine-1-butanoic acid, as analyzed by high-performance liquid chromatography (HPLC). In some embodiments, the peracetylgalactosamine-1-pentanoic acid of Formula A obtained from the disclosed methods comprises an amount of peracetylgalactosamine-1-butanoic acid that is below the level of detection, as analyzed by HPLC.

The embodiments of this disclosure and the examples demonstrate that GalNAc C5 linker can be synthesized using a vegetal sourced D-glucosamine instead of animal sourced D-galactosamine. The disclosure also demonstrates that it is possible to reduce the M-14 impurity species in the synthesized GalNAc C5 linker by using 5-hexen-1-ol which has low content of 4-hexen-1-ol.

### EXAMPLES

### Example 1. Synthetic Route of Vegetal GalNAc C5 Linker (Design Strategy “Route 1”)

Starting from vegetal glucosamine hydrochloride, a synthetic route according to design strategy “Route 1” is designed and depicted in FIG. 3. The first half of the route is to invert C-4 configuration of a derivatized D-glucosamine intermediate, leading into a derivatized D-galactosamine intermediate, then the subsequent synthetic reactions produce the target GalNAc C5 linker. Briefly, D-Glucosamine hydrochloride is acetylated to give the per-acetylated, derivatized D-glucosamine intermediate of formula 1 (“Intermediate 1”) in reaction “Step 1,” whose anomeric carbon is treated with trimethylsilyl trifluoromethanesulfonate (TMSOTf) in reaction “Step 2” to form the activated oxazoline, derivatized D-glucosamine intermediate of formula 2 (“Intermediate 2”), which is in turn readily glycosylated with 5-hexen-1-ol in reaction “Step 3” to afford the derivatized D-glucosamine intermediate of formula 3 (“Intermediate 3”). Hydrolysis of acetates of Intermediate 3 in reaction “Step 4” liberates three hydroxy groups at C3, C4 and C6 positions in the derivatized D-glucosamine intermediate of formula 4 (“Intermediate 4”). Pivoroyl chloride is then introduced in reaction “Step 5” to acylate selectively the hydroxyl groups at C3 and C6 positions, leaving the hydroxyl at C4 position intact in the derivatized D-glucosamine intermediate of formula 5 (“Intermediate 5”). That hydroxyl group at C4 position is then acylated using triflic anhydride in reaction “Step 6,” which is spontaneously converted to derivatized D-galactosamine intermediate of formula 6 (“Intermediate 6”) via an intramolecular migration of the pivaroyl group from C3 to C4 position, causing an inversion of the chiral configuration at C4 position. Hydrolysis of pivaroic esters in reaction “Step 7” leads to the derivatized D-galactosamine intermediate of formula 7 (“Intermediate 7”), whose hydroxyl groups are then acetylated in reaction “Step 8” to afford the derivatized D-galactosamine intermediate of formula 8 (“Intermediate 8”). An RuCl₃ catalyzed oxidation of the olefin using sodium periodate in reaction “Step 9” finally afford the peracetylgalactosamine-1-pentanoic acid of Formula A (“GalNAc C5 Linker”). The structure of this product is the same as an authentic reference standard produced using animal sourced D-galactosamine as starting material, characterized by means of HPLC co-elution, LC-MS, ¹H-NMR.

### 1. Description of Synthesis Procedures

i. Step 1: Synthesis of Intermediate 1 (2-Acetamido-1,3,4,6-tetra-O-acetyl-2-deoxy-α/β-D-glucopyranoside)

The reactor was charged pyridine (10 volume equivalents), D-glucosamine hydrochloride (1.0 volume equivalent), and dimethylpyridine (DMAP, 0.02 volume equivalent). The mixture was mixed and cooled to below 10° C. To this cooled mixture, acetic anhydride (7.0 volume equivalents) was added while stirring. Raised the temperature to 50° C. and stirred for 10 hours. An aliquot of the reaction mixture was analyzed on HPLC to monitor the remaining D-glucosamine. When the reaction was complete, the reaction mixture was added into 1 N HCl (5 volume equivalents), then was extracted using ethyl acetate (EA). The organic layer was collected, and the aqueous layer was extracted with fresh EA (5 volume equivalents). The organic layers were then combined, washed twice using 1 N HCl (5 volume equivalents each), once with saturated NaHCO₃ (5 volume equivalents), twice with 20% NaCl (5 volume equivalents each), concentrated down to about 3 volume equivalents. The concentrated EA solution was charged into methyl tert-butyl ether (MTBE, 20 volume equivalents), stirred for 2 hours to allow precipitation to form. Filtered to collect solid, rinsed the solid using MTBE (1 volume equivalent). The collected solid was then mixed with n-heptane (1 volume equivalent) to make a slurry. Filtered and collected the solid which was dried to provide the derivatized D-glucosamine intermediate of formula 1 (“Intermediate 1” in FIG. 3).

ii. Step 2: Synthesis of Intermediate 2 (2-Methyl-(3,4,6-tri-O-acetyl-1,2-dideoxy-α-D-glucopyrano)-[2,1-d]-2-oxazoline) (2-Methyl oxazoline)

The crude Intermediate 1 (2-Acetamido-1,3,4,6-tetra-O-acetyl-2-deoxy-α/β-D-glucopyranoside) was dissolved in 5 volume equivalents of acetonitrile (ACN) in reactor, and TMSOTf (1.3 volume equivalents) was added and mixed at room temperature. The whole mixture was heated to about 75° C., stirred for 2 hours. ACN was removed under vacuum, and the residue was taken up in dichloromethane (DCM, 5 volume equivalents), then added into pre-cooled (0-5° C.) saturated NaHCO₃ (9 volume equivalents), stirred for 30 minutes. After phases were separated, the aqueous layer was extracted with fresh DCM (3 volume equivalents), and the organic phases were combined. The organic solution was concentrated down to about 2 volume equivalents, and diluted with 5 volume equivalents of DCM, and concentrated it again down to about 2 volume equivalents. The dilution and concentration were repeated until Karl Fisher test gave moisture level less than 0.1%. The solution of this crude derivatized D-glucosamine intermediate of formula 2 (2-Methyl-(3,4,6-tri-O-acetyl-1,2-dideoxy-α-D-glucopyrano)-[2,1-d]-2-oxazoline; “Intermediate 2” in FIG. 3) was used directly in the next step of reaction, i.e., reaction “Step 3.”

iii. Step 3: Synthesis of Intermediate 3 (5-Hexenyl 2-acetamido-3,4,6-tri-O-acetyl-2-deoxy-β-D-glucopyranoside)

The concentrated solution of crude Intermediate 2 (2-Methyl-(3,4,6-tri-O-acetyl-1,2-dideoxy-α-D-glucopyrano)-[2,1-d]-2-oxazoline) was diluted further with 5 volume equivalents of DCM, and 5-hexen-1-ol was added at room temperature. The mixture was then cooled (0-5° C.) and TMSOTf (0.5 volume equivalent) was added. The mixture was allowed to warm to room temperature and stirred for 5 hours. The reaction mixture was then charged into pre-cooled water (10 volume equivalents, 0-5° C.). The collected organic layer was washed twice using 20% NaCl (5 volume equivalents each). The organic layer was filtered through a Magnesol pad (2 weight equivalents), followed by rinsed using tetrahydrofuran (15 volume equivalents). The collected solution was concentrated to about 2 volume equivalents, mixed with 5 volume equivalents of MTBE, concentrated again down to 2 volume equivalents. Then n-heptane (1 volume equivalent) was added, stirred for 2 hours to allow precipitate to form, which was then filtered, collected, and dried to produce dry solid product of the derivatized D-glucosamine intermediate of formula 3 (5-Hexenyl 2-acetamido-3,4,6-tri-O-acetyl-2-deoxy-β-D-glucopyranoside; “Intermediate 3” in FIG. 3).

iv. Step 4: Synthesis of Intermediate 4 (5-Hexenyl-N-acetyl-β-D-glucosamine)

The solid Intermediate 3 (5-Hexenyl 2-acetamido-3,4,6-tri-O-acetyl-2-deoxy-β-D-glucopyranoside; 1.0 volume equivalent) was dissolved in methanol (MeOH, 15 volume equivalents) in reactor. A solution of sodium methoxide (MeONa) in MeOH (30%, wt/v) is added and the temperature was maintained below 30° C. Stirred for 2 hours at room temperature until full consumption of Intermediate 3. Amberlyst 15 (1.5 weight equivalents) was added and stirred for 10 minutes. The mixture was filtered, and the cake was rinsed using MeOH (2 volume equivalents), the collected filtrate was concentrated to about 3 volume equivalents. The concentrated solution was mixed with MTBE (5 volume equivalents), stirred for 30 minutes, then charged n-heptane and stirred for 2 hours to allow precipitates for form. The solid was filtered and collected, which was dried to afford the derivatized D-glucosamine intermediate of formula 4 (5-Hexenyl-N-acetyl-β-D-glucosamine; “Intermediate 4” in FIG. 3).

v. Step 5: Synthesis of Intermediate 5 (5-Hexenyl 2-acetamido-3,6-di-O-pivaloyl-2-deoxy-β-D-glucopyranoside)

Intermediate 4 (5-Hexenyl-N-acetyl-β-D-glucosamine; 1.0 volume equivalent) was dissolved in pyridine (10 volume equivalents) in reactor, and cooled to about −5-0° C. Pivaloyl chloride (PivCl, 2.5 volume equivalents) was added dropwise into the mixture while stirring. After the addition was finished, the reaction mixture was warmed to about 20° C., stirred at least 10 hours to assure the completion of esterification. To the reaction mixture was then charged DCM (20 volume equivalents) and water (20 volume equivalents) to perform extraction while maintaining temperature lower than 20° C. After phase separation, the organic layer was collected and washed with 3N HCl three time using 10 volume equivalents each time, followed by washing once with saturated NaHCO₃ (10 volume equivalents) and twice with saturated NaCl (10 volume equivalents each). The organic layer was the concentrated to dryness to afford the derivatized D-glucosamine intermediate of formula 5 (5-Hexenyl 2-acetamido-3,6-di-O-pivaloyl-2-deoxy-β-D-glucopyranoside; “Intermediate 5” in FIG. 3).

vi. Step 6: Synthesis of Intermediate 6 (5-Hexenyl 2-acetamido-4,6-di-O-pivaloyl-2-deoxy-β-D-galactopyranoside)

Crude product of Intermediate 5 (5-Hexenyl 2-acetamido-3,6-di-O-pivaloyl-2-deoxy-β-D-glucopyranoside; 1.0 volume equivalent) was dissolved in DCM (7 volume equivalents) and pyridine (2.0 volume equivalents) in reactor, and cooled to about −30° C. Triflic anhydride (1.2 volume equivalents) was added dropwise into the mixture. After the addition was finished, continued to stir for another 2 hours at that temperature. Water (2 volume equivalents) was added to quench the reaction while the temperature was maintained below 20° C. After the completion of water addition, the reaction mixture was warmed to about 45° C., stirred for at least 5 hours to assure the completion of SN2 migration of pivaroyl group from C3 to C4 position. The mixture was concentrated to about 5 volume equivalents, then EA (10 volume equivalents) and water (5 volume equivalents) were added to perform extraction with temperature lower than 20° C. The collected organic layer was washed three times using 3N HCl (5 volume equivalents each), once using saturated NaHCO₃ (5 volume equivalents), twice using saturated NaCl (5 volume equivalents). The washed organic layer was then concentrated to dryness to afford crude derivatized D-galactosamine intermediate of formula 6 (5-Hexenyl 2-acetamido-4,6-di-O-pivaloyl-2-deoxy-β-D-galactopyranoside; “Intermediate 6” in FIG. 3).

vii. Step 7: Synthesis of Intermediate 7 (5-Hexenyl-N-acetyl-β-D-galactosamine)

Crude product of Intermediate 6 (5-Hexenyl 2-acetamido-4,6-di-O-pivaloyl-2-deoxy-β-D-galactopyranoside; 1.0 volume equivalent) was dissolved in MeOH (15 volume equivalents) in reactor. 0.85 volume equivalent of MeONa in MeOH (30% weight/volume) was added while maintaining temperature below 30° C. After the addition was finished, continued to stir for another 2 hours at around 25° C. until Intermediate 6 is less than 2% on HPLC. Amberlyst 15 (1.5 weight equivalents) was added to the mixture, stirred for 10 minutes. Filtered and concentrated the collected filtrate to about 3 volume equivalents. To the concentrated solution was added slowly MTBE (5 volume equivalents), stirred for 30 minutes, followed by addition of n-heptane (10 volume equivalents). Stirred for another 2 hours to allow precipitates for form. Solid cake was filtered, collected, and rinsed using n-heptane (2 volume equivalents). The cake was dried to afford the derivatized D-galactosamine intermediate of formula 7 (5-Hexenyl-N-acetyl-β-D-galactosamine; “Intermediate 7” in FIG. 3).

viii. Step 8: Synthesis of Intermediate 8 (5-Hexenyl 2-acetamido-3,4,6-tri-O-acetyl-2-deoxy-β-D-galactopyranoside)

Crude product of Intermediate 7 (5-Hexenyl-N-acetyl-β-D-galactosamine; 1.0 volume equivalent) was dissolved in pyridine (7 volume equivalents) with DMAP (0.02 volume equivalent) in reactor. The mixture was cooled below 15° C. and acetic anhydride (5 volume equivalents) was added. After the addition was complete, the reaction mixture was warmed to room temperature and stirred for 10 hours to assure the completion of acetylation. DCM (20 volume equivalents) and water (10 volume equivalents) were charged into the reactor to quench the reaction and perform extraction. The organic layer was washed three times with 3N HCl (10 volume equivalents each), once with saturated NaHCO₃ (10 volume equivalents), twice with saturated NaCl (10 volume equivalents), then concentrated to about 2 volume equivalents. MTBE (5 volume equivalents) was added, mixed, and concentrated down to 2 volume equivalents. n-heptane (2 volume equivalents) was added, mixed, and stirred for 2 hours to allow precipitations to form. Filtered the mixture and collected the solid which was dried to afford the derivatized D-galactosamine intermediate of formula 8 (5-Hexenyl 2-acetamido-3,4,6-tri-O-acetyl-2-deoxy-β-D-galactopyranoside; “Intermediate 8” in FIG. 3).

ix. Step 9: Synthesis of GalNAc-C5-Linker (5-[[3,4,6-tri-O-acetyl-2-acetamido-2-deoxy-β-D-galactopyranosyl]oxy]-pentanoic acid)

Crude product of Intermediate 8 (5-Hexenyl 2-acetamido-3,4,6-tri-O-acetyl-2-deoxy-β-D-galactopyranoside; 1.0 volume equivalent) was mixed in reactor with acetonitrile (4 volume equivalents), DCM (4 volume equivalents), water (6 volume equivalents), and RuCl₃·3H₂O (0.013 volume equivalent). The mixture was cooled to 0-5° C., and NaIO₄ (4.1 volume equivalents) was added portion by portions. Stirred for 2 hours until the completion of the reaction. The reaction mixture was filtered through Diatomite (1 weight equivalent) pad. The pH of the filtrated was adjusted to about 8 using saturated NaHCO₃. Stirred for 30 minutes and separated the phases to collect the aqueous layer. The aqueous layer was washed four time using DCM (3 volume equivalents each). The pH was adjusted to about 2 using citric acid, and extraction was performed using DCM six times (5 volume equivalents each), the collected organic layers were combined and concentrated down to about 3 volume equivalents. MTBE (8 volume equivalents) was added in two equal portions, mixed and concentrated to about 3 volume equivalents. MTBE (4 volume equivalents) was added again, stirred for 2 hours to allow precipitates to form. Filtered to collect the solid which was dried to afford target peracetylgalactosamine-1-pentanoic acid of Formula A (“GalNAc C5 Linker” in FIG. 3).

### 2. Characterization of Intermediates and Final Product

Intermediates and the final product “GalNAc C5 Linker” were characterized by HPLC, LC-MS, and ¹H-NMR analyses.

i. Intermediate 4 (5-Hexenyl-N-acetyl-β-D-glucosamine) and Intermediate 7 (5-Hexenyl-N-acetyl-β-D-galactosamine)

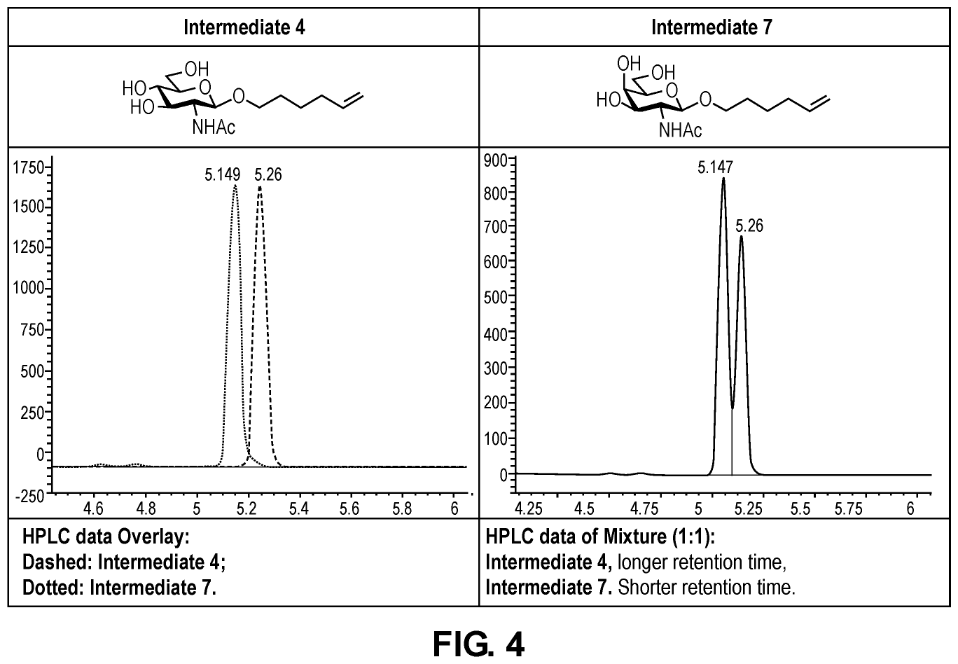

The glycosylated Intermediate 4 and Intermediate 7 were analyzed using HPLC. As shown in FIG. 4, HPLC analysis showed different retention times between Intermediate 4 and Intermediate 7, indicating the conversion of C4 chiral configurations.

ii. Intermediate 8 (5-Hexenyl 2-acetamido-3,4,6-tri-O-acetyl-2-deoxy-β-D-galactopyranoside)

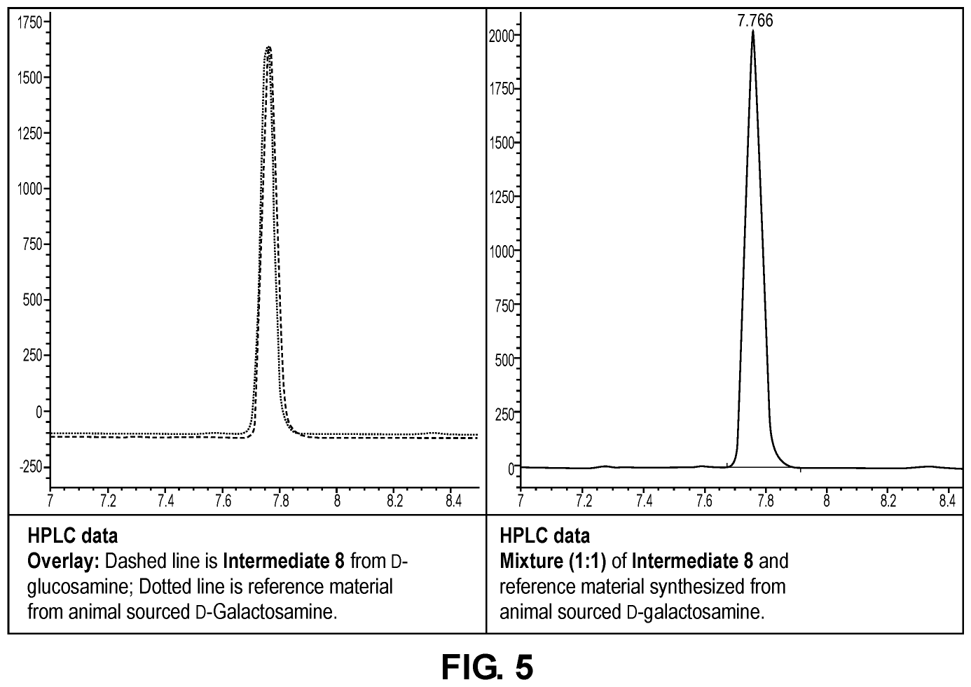

Intermediate 8 was compared to synthetic reference material synthesized using animal sourced D-galactosamine. As shown in FIG. 5, HPLC and ¹H-NMR analyses confirmed that this product was identical to synthetic reference material synthesized using animal sourced D-galactosamine.

iii. GalNAc C5 Linker (5-[[3,4,6-tri-O-acetyl-2-acetamido-2-deoxy-β-D-galactopyranosyl]oxy]-pentanoic acid)

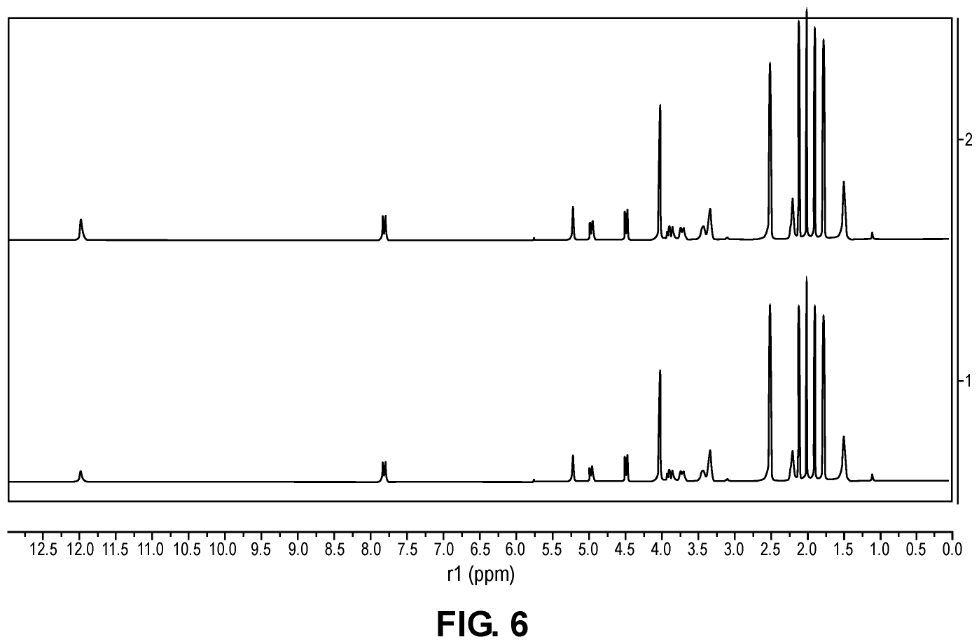

The final product “GalNAc C5 Linker” (5-[[3,4,6-tri-O-acetyl-2-acetamido-2-deoxy-β-D-galactopyranosyl]oxy]-pentanoic acid) was compared to synthetic reference material synthesized using animal sourced D-galactosamine. As shown in FIG. 6, HPLC and ¹H-NMR analyses confirm that the GalNAc C5 linker synthesized using vegetal-sourced D-glucosamine was identical to GalNAc C5 linker synthesized using animal sourced D-galactosamine.

### 3. Elimination of Impurity Species

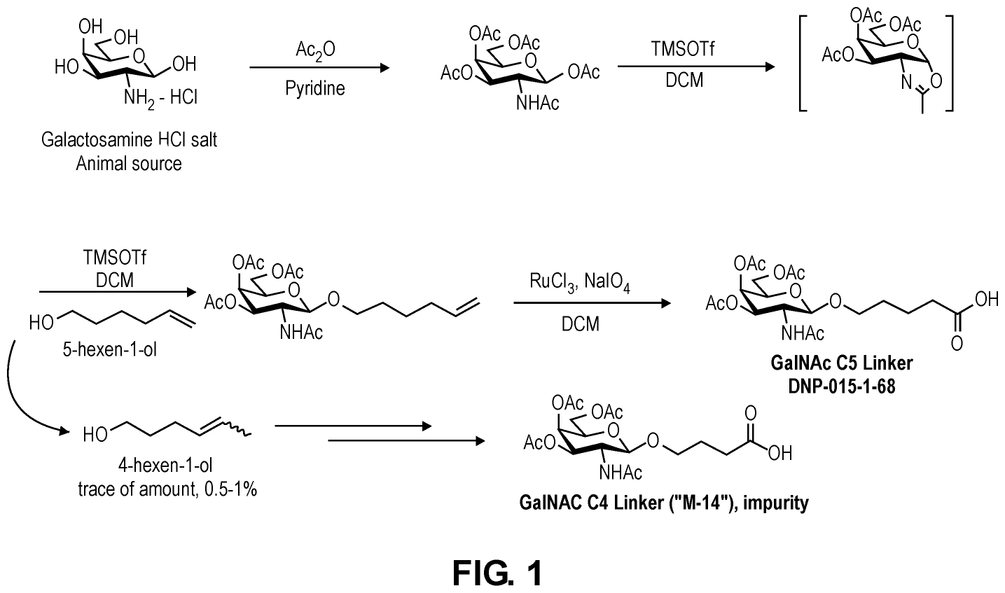

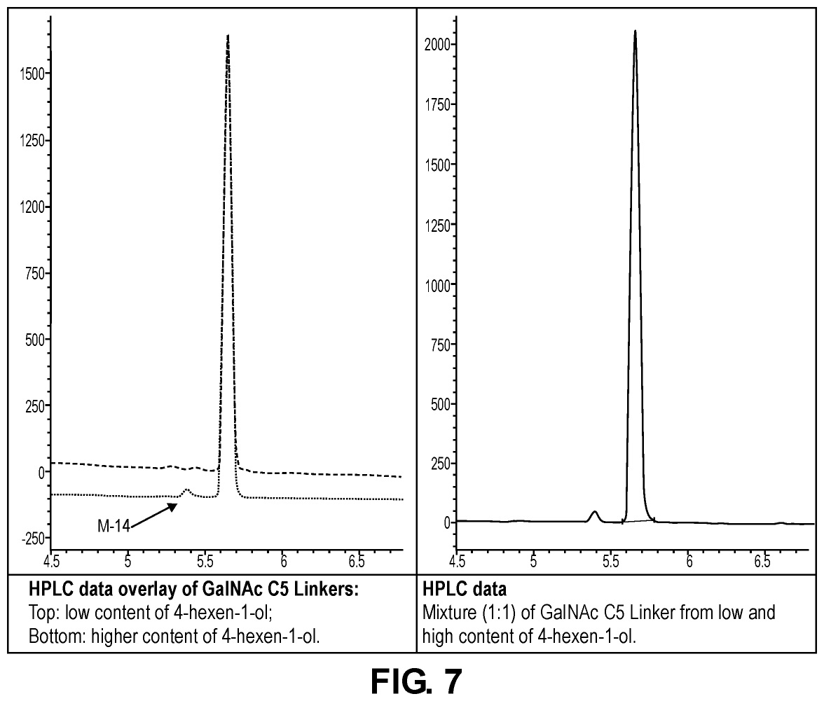

There has been a persistent impurity species, namely peracetylgalactosamine-1-butanoic acid (or GalNAc Linker or “M-14”), in the amidite product, detectable up to 1% on HPLC, which may be incorporated into a drug substance. As shown in FIG. 1, a contaminating impurity, 4-hexen-1-ol, in raw material 5-hexen-1-ol is the source of M-14 impurity species in GalNAc C5 Linker. It has been observed in the past that “M-14” cannot be fully removed when the impurity 4-hexen-1-ol is at higher levels (roughly 0.4% and higher). When the 4-hexen-1-ol content in raw material 5-hexen-1-ol is below a certain limit, such as <0.2%, an isolation and purification process of the final carboxylic acid, as described above in connection with Reaction “Step 9,” was demonstrated to have capacity to remove the M-14 impurity species to near or below the detection or quantification limits of the HPLC method. As shown in FIG. 7, the HPLC analysis showed that the M-14 impurity species was visible in the GalNAc C5 linker synthesized using raw material 5-hexen-1-ol with higher content of 4-hexen-1-ol but not in the GalNAc C5 linker synthesized using raw material 5-hexen-1-ol with low content of 4-hexen-1-ol.

In addition to the M-14 impurity, it has also been demonstrated that this synthetic route can also diminish other impurities such as α-isomer and epimers.

### 4. Conclusions

This example demonstrates that GalNAc C5 linker can be synthesized using a vegetal sourced D-glucosamine instead of animal sourced D-galactosamine. It also demonstrates that it is possible to reduce the M-14 impurity species in the synthesized GalNAc C5 linker by using 5-hexen-1-ol which has low content of 4-hexen-1-ol.

### Example 2. Alternate Synthetic Route of GalNAc C5 Linker (Design Strategy “Route 2”)

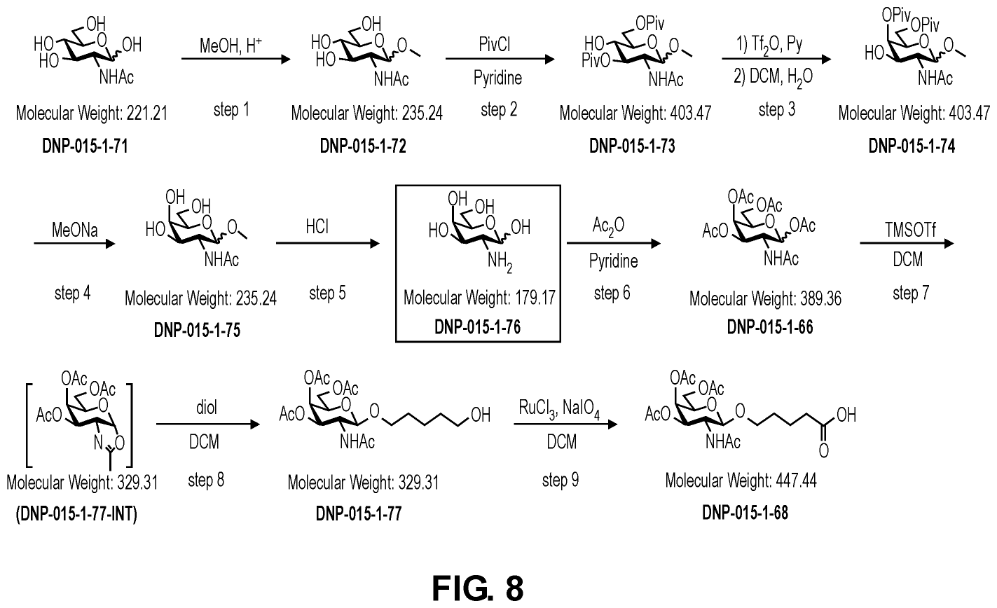

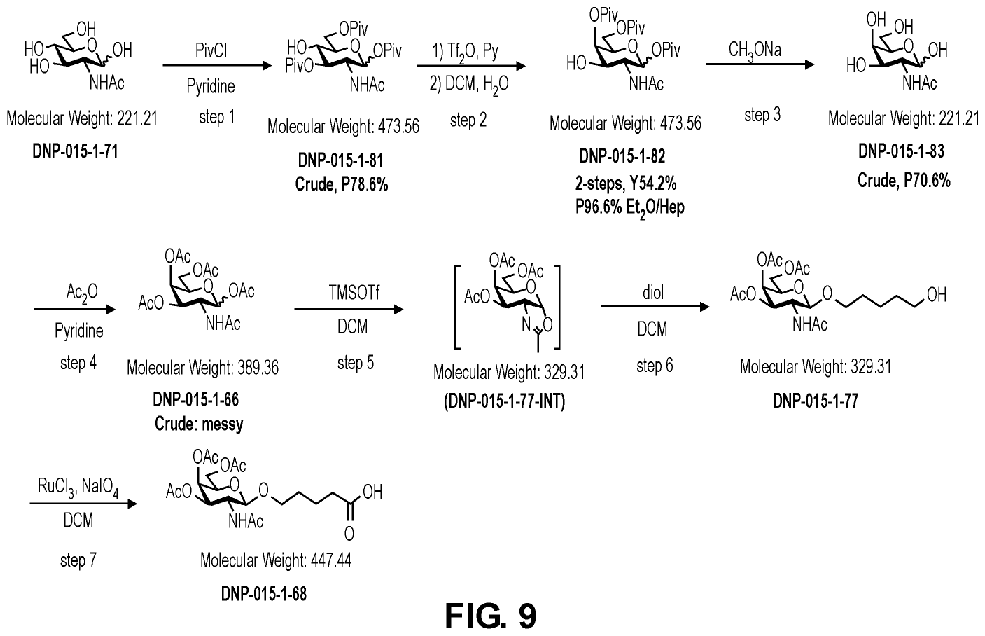

The synthetic route described in Example 1 (design strategy “Route 1”) introduces glycosylation early in the synthetic process followed by C4 inversion. An alternate synthetic route, such as design strategy “Route 2” depicted in FIG. 2B, may first introduce C4 inversion followed by glycosylation. This alternate synthetic route allows making galactosamine as a stock-up intermediate. It also makes it possible to use different C5 linkers for glycosylation. Two synthetic schemes according to the concept of design strategy “Route 2” are depicted in FIG. 8 and FIG. 9.

The design strategy “Route 2” uses N-acetylglucosamine as the starting material and comprises at least the following steps:
a) subjecting N-acetylglucosamine to hydrolysis followed by selective acetylation to obtain a derivatized D-glucosamine intermediate of formula 9:

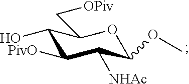

b) subjecting the derivatized D-glucosamine intermediate of formula 9 to triflation leading to an inversion of the chiral configuration at C4 position to obtain a derivatized D-galactosamine intermediate of formula 10:

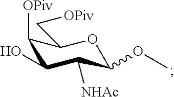

c) subjecting the derivatized D-galactosamine intermediate of formula 10 to hydrolysis followed by deacetylation to obtain a derivatized D-galactosamine intermediate of formula 12:

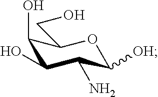

and
d) subjecting the derivatized D-galactosamine intermediate of formula 12 to acetylation followed by glycosylation in presence of diol to obtain a derivatized D-galactosamine intermediate of formula 15:

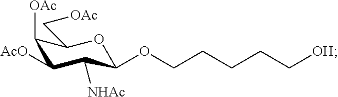

and
e) subjecting the derivatized D-galactosamine intermediate of formula 15 to oxidation in presence of a catalyst thereby producing the peracetylgalactosamine-1-pentanoic acid of Formula A.

N-acetylglucosamine is hydrolyzed by sodium methoxide, wherein the hydrolysis of N-acetylglucosamine is performed in methanol. The hydrolysis of N-acetylglucosamine leads to the production of a derivatized D-glucosamine intermediate of formula 19:

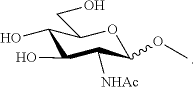

To obtain the derivatized D-glucosamine intermediate of formula 9, the derivatized D-glucosamine intermediate of formula 19 is subjected to selective protection as pivaloyl esters at C3 and C6 positions. This selective protection is conducted by pivaloyl chloride. This selective protection by pivaloyl chloride is performed in the presence of pyridine.

The triflation followed by the C4 inversion of the derivatized D-glucosamine intermediate of formula 9, leading to the production of the derivatized D-galactosamine intermediate of formula 10, is mediated by triflic anhydride. The oxygen atom at the C4 position, also referred to as the “O-4 position,” is activated by triflate to form an intermediate O-4 triflate followed by an intramolecular attack of the intermediate O-4 triflate by the pivaloyl group at the C3 position, also referred to as the “O-3-pivaloyl group,” resulting in an in-situ displacement of the triflate to yield the derivatized D-galactosamine intermediate of formula 10. This triflation followed by C4 inversion is performed in the presence of pyridine and DCM.

The hydrolysis of the derivatized D-galactosamine intermediate of formula 10 is performed using sodium methoxide in methanol. The hydrolysis of the derivatized D-galactosamine intermediate of formula 10 leads to the production of a derivatized D-galactosamine intermediate of formula 11:

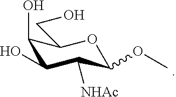

To obtain the derivatized D-galactosamine intermediate of formula 12, the derivatized D-galactosamine intermediate of formula 11 is subjected to deacetylation. This deacetylation is performed using hydrogen chloride.

The acetylation of the derivatized D-galactosamine intermediate of formula 12 is performed in the presence of acetic anhydride catalyzed by pyridine and 4-dimethylaminopyridine (DMAP). The acetylation of the derivatized D-galactosamine intermediate of formula 12 by acetic anhydride leads to the production of a derivatized D-galactosamine intermediate of formula 13:

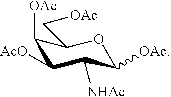

The derivatized D-galactosamine intermediate of formula 13 is subjected to a reaction with TMSOTf. This reaction with TMSOTf is performed in the presence of acetonitrile (CAN). The reaction of the derivatized D-galactosamine intermediate of formula 13 with TMSOTf leads to the production of a derivatized D-galactosamine intermediate of formula 14:

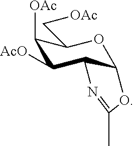

The derivatized D-galactosamine intermediate of formula 14 is extracted using dichloromethane (DCM) and subjected to glycosylation by diol. The glycosylation of the derivatized D-galactosamine intermediate of formula 14 by diol leads to the production of the derivatized D-galactosamine intermediate of formula 15.

The oxidation of the derivatized D-galactosamine intermediate of formula 15 by sodium periodate is catalyzed by ruthenium chloride, wherein the oxidation of derivatized D-galactosamine intermediate of formula 15 is performed in the presence of acetonitrile, DCM, and water to produce the peracetylgalactosamine-1-pentanoic acid or GalNAc C5 linker.

In the synthetic scheme shown in FIG. 8, N-acetylglucosamine (“DNP-015-1-71”) is hydrolyzed by MeOH in step 1 to produce a derivatized D-glucosamine intermediate of formula 19 (“DNP-015-1-72”), which is then subjected to selective protection as pivaloyl esters at C3 and C6 positions by pivaloyl chloride in the presence of pyridine in step 2 to produce a derivatized glucosamine intermediate of formula 9 (“DNP-015-10-73”). Subjecting DNP-015-10-73 to triflation followed by a SN2 migration of a pivaroyl group from C3 to C4 position by triflic anhydride in the presence of pyridine in step 3 allows the production of a derivatized D-galactosamine intermediate of formula 10 (“DNP-015-1-74”). Hydrolysis of DNP-015-1-74 by sodium methoxide in step 4 leads to a derivatized D-galactosamine intermediate of formula 11 (“DNP-015-1-75”), which is then deacetylated in step 5 to produce a derivatized D-galactosamine intermediate of formula 12 (“DNP-015-1-76” or galactosamine). DNP-015-1-76 is then acetylated by acetic anhydride in the presence of pyridine in step 6 to produce a derivatized D-galactosamine intermediate of formula 13 (“DNP-015-1-66”), which is then reacted with TMSOTf in step 7 to produce a derivatized D-galactosamine intermediate of formula 14 (“DNP-015-1-77-INT”). Glycosylation of DNP-015-1-77-INT in the presence of a diol and DCM in step 8 leads to the production of a derivatized D-galactosamine intermediate of formula 15 (“DNP-015-1-77”). RuCI₃ catalyzed oxidation of DNP-015-1-77 by sodium periodate in step 9 thereby produces the peracetylgalactosamine-1-pentanoic acid or GalNAc C5 linker (“DNP-015-1-68).

An alternative synthetic route based on design strategy “Route 2” may perform selective acetylation by pivaloyl chloride directly on N-acetylglucosamine without subjecting it to hydrolysis. This synthetic route of the disclosed methods based on design strategy “Route 2” may comprise at least the following steps:
a) reacting N-acetylglucosamine with pivaloyl chloride to obtain a derivatized D-glucosamine intermediate of formula 16:

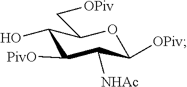

b) reacting the derivatized D-glucosamine intermediate of formula 16 with triflic anhydride to obtain a derivatized D-galactosamine intermediate of formula 17:

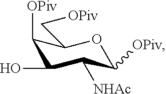

wherein the derivatized D-glucosamine intermediate of formula 16 is activated at O-4 position by triflate to form an intermediate O-4 triflate followed by an intramolecular attack of the intermediate O-4 triflate by the O-3-pivaloyl group resulting in an in-situ displacement of the triflate to yield the derivatized D-galactosamine intermediate of formula 17;
c) subjecting the derivatized D-galactosamine intermediate of formula 17 to hydrolysis followed by acetylation to obtain the derivatized D-galactosamine intermediate of formula 13:

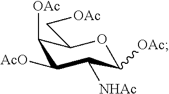

d) reacting the derivatized D-galactosamine intermediate of formula 13 with TMSOTf to obtain the derivatized D-galactosamine intermediate of formula 14:

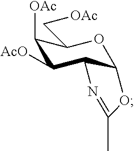

e) subjecting the derivatized D-galactosamine intermediate of formula 14 to glycosylation by a diol followed by oxidation in the presence of a catalyst thereby producing the peracetylgalactosamine-1-pentanoic acid of Formula A.

The derivatized D-galactosamine intermediate of formula 17 is hydrolyzed by sodium methoxide in methanol. The hydrolysis of the derivatized D-galactosamine intermediate of formula 17 leads to the production of a derivatized D-galactosamine intermediate of formula 18:

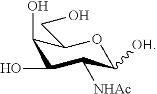

To obtain the derivatized D-galactosamine intermediate of formula 13, the derivatized D-galactosamine intermediate of formula 18 is subjected to acetylation by acetic anhydride catalyzed by pyridine and 4-dimethylaminopyridine (DMAP).

The reaction of the derivatized D-galactosamine intermediate of formula 13 with TMSOTf is performed in the presence of acetonitrile (CAN).

The derivatized D-galactosamine intermediate of formula 14 is extracted using DCM. In some embodiments, the glycosylation of the derivatized D-galactosamine intermediate of formula 14 by a diol leads to the production of the derivatized D-galactosamine intermediate of formula 15:

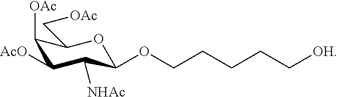

To obtain the peracetylgalactosamine-1-pentanoic acid of Formula A, the derivatized D-galactosamine intermediate of formula 15 is subjected to an oxidation in the presence of a catalyst. The derivatized D-galactosamine intermediate of formula 15 is subjected to an oxidation by sodium periodate catalyzed by ruthenium chloride in the presence of acetonitrile, DCM, and water to produce the peracetylgalactosamine-1-pentanoic acid or GalNAc C5 linker.

In the synthetic scheme shown in FIG. 9, N-acetylglucosamine (“DNP-015-1-71”) is subjected to selective protection as pivaloyl esters at C3 and C6 positions by pivaloyl chloride in the presence of pyridine in step 1 to produce a derivatized glucosamine intermediate of formula 16 (“DNP-015-1-81”). Subjecting DNP-015-1-81 to triflation followed by a SN2 migration of a pivaroyl group from C3 to C4 position by triflic anhydride in the presence of pyridine in step 2 allows the production of a derivatized D-galactosamine intermediate of formula 17 (“DNP-015-1-82”). Hydrolysis of DNP-015-1-82 by sodium methoxide in step 3 leads to a derivatized D-galactosamine intermediate of formula 18 (“DNP-015-1-83”), which is then acetylated by acetic anhydride in the presence of pyridine in step 4 to produce a derivatized D-galactosamine intermediate of formula 13 (“DNP-015-1-66”). Reacting DNP-015-1-66 with TMSOTf in step 5 to produce a derivatized D-galactosamine intermediate of formula 14 (“DNP-015-1-77-INT”). Glycosylation of DNP-015-1-77-INT in the presence of a diol and DCM in step 6 leads to the production of a derivatized D-galactosamine intermediate of formula 15 (“DNP-015-1-77”). RuCl₃ catalyzed oxidation of DNP-015-1-77 by sodium periodate in step 7 thereby produces the peracetylgalactosamine-1-pentanoic acid or GalNAc C5 linker (“DNP-015-1-68).

The synthetic procedures disclosed in FIG. 8 and FIG. 9 based on the design strategy “route 2” faced many problems. The reactions were messy, difficult to purify and produced very low yield for the intermediates as compared to the robust synthetic procedure based on design strategy “route 1”.

### Example 3. Strategies for Elimination of Impurities

The presence of impurities, such as M-14, α-isomer and epimers, in the current synthetic route using galactosamine as the starting material has been persistent. Strategies for eliminating these impurities were investigated. As shown in FIG. 1, trace amount of 4-hexen-1-ol present in raw material 5-hexen-1-ol is the source of one of the impurity species, namely M-14, in the synthesis of GalNAc C5 linker. It has been observed in the past that M-14 cannot be fully removed when the impurity 4-hexen-1-ol in raw material 5-hexen-1-ol is at higher levels (roughly 0.4% and higher). Thus, the first strategy to eliminate the M-14 impurity is to control the content of 4-hexen-1-ol in 5-hexen-1-ol raw material. To this end, high quality of 5-hexen-1-ol raw material was obtained from multiple suppliers and analyzed for 4-hexen-1-ol (Table 1).

TABLE 1
Selection of commercial sources of 5-hexen-1-ol.

| | Area%, GC | | | |
|---|---|---|---|---|
| Vendor | 5-hexen-1-ol 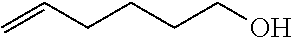 | 4-hexen-1- ol(E/Z) 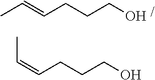 | 6-hepten-1-ol 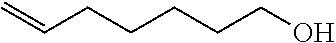 | 7-octen-1-ol 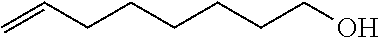 |
| Vendor A, lot a | 99.3% | 0.03% | ND | ND |
| Vendor A, lot b | 96.8% | 0.11% | ND | ND |
| Vendor B, lot a | 99.4% | 0.15% | ND | ND |
| Vendor B, lot b | 99.3% | 0.03% | ND | ND |
| Vendor C, lot a | 99.6% | 0.14% | ND | ND |

Next, a strategy of selective removal of M-14 and α-isomer through an isolation and purification process of the final product was tested. This isolation and purification process includes a first partition using saturated sodium bicarbonate, followed by a second partition using citric acid. An example of this isolation and purification process is described in Reaction “Step 9” of Example 1, and the amount of GalNAc C5 linker (“Target”), M-14 impurity (“M-14”) and α-isomer present in each phase of each partition was determined by HPLC (Table 2). As shown in Table 2, it is possible to remove selectively M-14 and α-isomer through this isolation and purification process.

TABLE 2
Selective removal of M-14 and α-isomer.

| | | HPLC Analysis, area % | | |
|---|---|---|---|---|
| Operation Stages | | M-14 | Target | α-Isomer |
| Operation | HPLC samples | 5.1 | 5.6 | 6.0 |
| Reaction Mixture | IPC (1.5 h) | 1.3 | 93.1 | 0.9 |
| First Partition | Organic phase | 12.4 | 13.1 | 18.4 |
| | Alkaline aqueous phase | 1.1 | 97.8 | ND |
| Second partition | Organic phase | 1.1 | 97.0 | ND |
| | Acidic aqueous phase | 4.4 | 86.7 | ND |

A third strategy for reducing the M-14 impurity is to use alternative C5 linkers of high purity. Both 1,5-pentanediol and benzyl 5-hydroxypentanoate can be such alternative C5 linkers and using 1,5-pentanediol for glycosylation appears to lower the amount of M-14 and α-isomer impurities (data not shown). However, glycosylation requires a large molar excess of 1,5-pentanediol, which may present a purification challenge. Additionally, pure 1,5-pentanediol is not easy to obtain with consistent quality, which may introduce new impurity species.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the protein constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1. (canceled)

2. A method of producing peracetylgalactosamine-1-pentanoic acid of Formula A 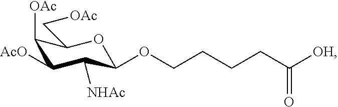
comprising using a vegetal source as a starting material.

3. The method of claim 2, wherein the starting material comprises D-glucosamine or D-glucosamine hydrochloride.

4. A method for producing peracetylgalactosamine-1-pentanoic acid of Formula A 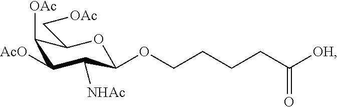
comprising:
a) subjecting D-glucosamine hydrochloride to acetylation by acetic anhydride to obtain a derivatized D-glucosamine intermediate of formula 1 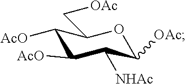
b) reacting the derivatized D-glucosamine intermediate of formula 1 with trimethylsilyl trifluoromethanesulfonate (TMSOTf) to obtain a derivatized D-glucosamine intermediate of formula 2 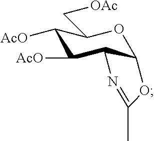
c) subjecting the derivatized D-glucosamine intermediate of formula 2 to glycosylation by 5-hexen-1-ol to obtain a derivatized D-glucosamine intermediate of formula 3 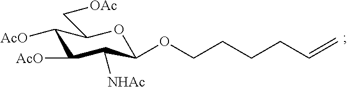
d) subjecting the derivatized D-glucosamine intermediate of formula 3 to hydrolysis by sodium methoxide to obtain a derivatized D-glucosamine intermediate of formula 4 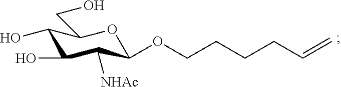
e) reacting the derivatized D-glucosamine intermediate of formula 4 with pivaloyl chloride to obtain a derivatized D-glucosamine intermediate of formula 5 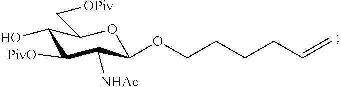
f) reacting the derivatized D-glucosamine intermediate of formula 5 with triflic anhydride to obtain a derivatized D-galactosamine intermediate of formula 6 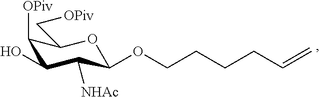
wherein the derivatized D-glucosamine intermediate of formula 5 is activated at O-4 position by triflate to form an intermediate O-4 triflate followed by an intramolecular attack of the intermediate O-4 triflate by the O-3-pivaloyl group resulting in an in-situ displacement of the triflate to yield the derivatized D-galactosamine intermediate of formula 6;
g) subjecting the derivatized D-galactosamine intermediate of formula 6 to hydrolysis by sodium methoxide to obtain a derivatized D-galactosamine intermediate of formula 7 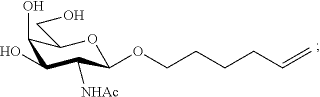
h) subjecting the derivatized D-galactosamine intermediate of formula 7 to acetylation by acetic anhydride to obtain a derivatized D-galactosamine intermediate of formula 8 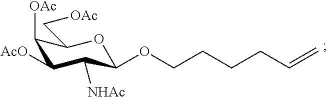
i) subjecting the derivatized D-galactosamine intermediate of formula 8 to oxidation by sodium periodate thereby producing the peracetylgalactosamine-1-pentanoic acid of Formula A.

5. The method of claim 4, wherein in step a) the acetylation of D-glucosamine hydrochloride by acetic anhydride is catalyzed by pyridine and 4-dimethylaminopyridine (DMAP).

6. The method of claim 4, wherein in step a) the derivatized D-glucosamine intermediate of formula 1 is extracted using ethyl acetate.

7. The method of claim 4, wherein in step b) the derivatized D-glucosamine intermediate of formula 2 is extracted using dichloromethane (DCM).

8. The method of claim 4, wherein in step c) the glycosylation of the derivatized D-glucosamine intermediate of formula 2 is performed in the presence of TMSOTf.

9. The method of claim 4, wherein in step d) the hydrolysis of the derivatized D-glucosamine intermediate of formula 3 is performed in methanol.

10. The method of claim 4, wherein in step e) the derivatized D-glucosamine intermediate of formula 4 is reacted with pivaloyl chloride in pyridine.

11. The method of claim 4, wherein in step e) the derivatized D-glucosamine intermediate of formula 5 is extracted using DCM and water.

12. The method of claim 4, wherein in step f) reacting triflic anhydride with the derivatized D-glucosamine intermediate of formula 5 is performed in the presence of pyridine and DCM.

13. The method of claim 4, wherein in step f) the derivatized D-galactosamine intermediate of formula 6 is extracted using ethyl acetate.

14. The method of claim 4, wherein in step g) the hydrolysis of the derivatized D-galactosamine intermediate of formula 6 is performed in methanol.

15. The method of claim 4, wherein in step h) the acetylation of the derivatized D-galactosamine intermediate of formula 7 is catalyzed by pyridine and DMAP.

16. The method of claim 4, wherein in step h) the derivatized D-galactosamine intermediate of formula 8 is extracted using DCM and water.

17. The method of claim 4, wherein in step i) the oxidation of the derivatized D-galactosamine intermediate of formula 8 is carried out in the presence of a catalyst.

18. The method of claim 17, wherein the catalyst is ruthenium chloride.

19. The method of claim 4, wherein in step i) the oxidation of the derivatized D-galactosamine intermediate of formula 8 is performed in the presence of acetonitrile, DCM, and water.

20. The method of claim 4, wherein in step i) the peracetylgalactosamine-1-pentanoic acid of Formula A is extracted using DCM.

21. (canceled)

22. The method of claim 4, wherein the 5-hexen-1-ol used for the glycosylation of the derivatized D-glucosamine intermediate of formula 2 in step c) contains less than about 0.2% of 4-hexen-1-ol.

23-25. (canceled)
